(19) United States
(12) Patent Application Publication
(10) Pub. No.: US 2014/0370054 A1
(43) Pub. Date: Dec. 18, 2014

(54) METHODS FOR INHIBITION OF TYPE 1 DIABETES

(71) Applicants: Zvi Laron, Tel Aviv (IL); Lester Shulman, Tel Aviv (IL)

(72) Inventors: Zvi Laron, Tel Aviv (IL); Lester Shulman, Tel Aviv (IL)

(21) Appl. No.: 14/304,958

(22) Filed: Jun. 15, 2014

Related U.S. Application Data
(60) Provisional application No. 61/834,905, filed on Jun. 14, 2013.

(51) Int. Cl. A61K 39/15

(52) U.S. Cl. CPC A61K 39/15
USPC 424/215.1

(57) ABSTRACT

This disclosure is directed to methods of inhibiting the development of type 1 diabetes in a subject, including methods of preventing development of type 1 diabetes. The disclosed methods involve pre-partum vaccination of future mothers against rotavirus. Methods of decreasing the prevalence of type 1 diabetes are also disclosed.

### CROSS REFERENCE TO RELATED APPLICATIONS

Benefit is claimed to U.S. Provisional Patent Application No. 61/834,905, which was filed on Jun. 14, 2013; the contents of which are incorporated by reference herein in their entirety.

### FIELD

This disclosure is directed to methods of inhibiting the development of type 1 diabetes in a subject, including methods of preventing development of type 1 diabetes. The disclosed methods involve pre-conception vaccination of future mothers against rotavirus. Methods of decreasing the prevalence of type 1 diabetes are also disclosed.

### BACKGROUND

Type 1 diabetes (T1D) is an autoimmune disease with both genetic and environmental risk factors contributing to its etiology. Specific autoantibodies directed to beta cell antigens, such as GAD65 (glutamic acid decarboxylase), are important markers of an ongoing autoimmune process involving the pancreatic beta cells. The incidence of T1D has been increasing annually throughout the world, especially in very young children (C. C. Patterson et al., Lancet, 373: 2027-2033, 2009).

Genetic susceptibility is conferred predominantly via HLA class II molecules, however, only 6% of individuals carrying the highest risk genotype developTiD. This suggests that one or more environmental factors play a role in T1D initiation and pathogenesis progression. According to von Herrath et al. (Nat Rev Microbiol, 1:151-157, 2003), the initial triggering events may be more specific than subsequent triggering events for disease pathogenesis. For example, an initial specific viral infection may trigger an autoimmune attack of pancreatic beta cells, which is then followed by other less specific immune stimulations. It is thought that the intensity and number of these events will determine the timing of the clinical unmasking of T1D (Laron Z et al., Am J Med Genet, 115, 2002).

The direct connection between rotavirus and T1D has been documented Honeyman, et al., Diabetes, 49: 1319-1324, 2000). Until the present disclosure however, it was not known if rotavirus immunity in a mother could affect the development of T1D in a subject as a result of rotavirus infection.

### SUMMARY

Provided herein are methods for inhibiting the development of type 1 diabetes mellitus in a subject which include vaccinating a future mother of the subject against at least one rotavirus prior to conception of the subject. Such vaccination will inhibit the occurrence of an autoimmunity event in the pregnant mother and developing fetus, which can lead to development of type 1 diabetes in the subject after birth.

Also provided herein are methods for decreasing the prevalence of type 1 diabetes mellitus, for example in a population. The methods include first determining if a woman of child-bearing age has been vaccinated against rotavirus; and prior to conception, vaccinating any non-vaccinated women against at least one rotavirus. Such vaccination will inhibit the occurrence of an autoimmunity event in the pregnant mother and any developing fetus, which can lead to development of type 1 diabetes in the subject after birth. Accordingly, the prevalence of the disease will be decreased.

The foregoing and other objects, features, and advantages will become more apparent from the following detailed description.

### DETAILED DESCRIPTION

### I. Abbreviations

ELISA Enzyme linked immunosorbent assay

T1D Type 1 diabetes mellitus

### II. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

Administration: The introduction of a composition into a subject, such as a vaccine, by a chosen route. Administration of a composition can be by any route known to one of skill in the art that is suitable for the administration of a vaccine, such as but not limited to a live viral vaccine or an attenuated viral vaccine. Modes of administration include and are not limited to, oral administration, topical administration, subcutaneous administration, and intramuscular routes. Administration can be local or systemic. Examples of local administration include, but intrathecal administration, or administration to the nasal mucosa or lungs by inhalational administration. Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Antibody: A polypeptide ligand comprising at least a light chain or heavy chain immunoglobulin variable region, which specifically recognizes and binds an epitope of an antigen, such as a rotavirus capsid protein or glycoprotein, or a fragment thereof Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W. H. Freeman & Co., New York, 1997.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. Vaccines against a microbe, such as a vaccine to produce immunity against at least one rotavirus, present microbe-specific antigens to a subject with the aim of developing immunity against the microbe.

Autoimmune disease: A disease resulting from an aberrant immune response, such as the production of antibodies or cytotoxic T cells specific for a self antigen or a subject's own cells or tissues. Autoimmune diseases include, but are not limited to, type 1 diabetes mellitus.

Child-bearing age: An age in which a woman is capable of becoming pregnant and carrying fetus to term, resulting in a live birth. Without being bound by a specific age, the child bearing age or girls and women begins with menarche and lasts until menopause.

Control: A reference standard. In particular examples a control sample is taken from a subject that is known not to have a disease or condition, or that is vaccinated for a disease. A difference between a test sample and a control can be an increase or conversely a decrease. The difference can be a qualitative difference or a quantitative difference, for example a statistically significant difference. In some examples, a difference is an increase or decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Detect: To determine if an agent (such as an antibody, for example an antibody specific to a particular rotavirus species or serotype) is present or absent. In some examples, this can further include quantification. As used herein, “detecting” the presence of an agent is synonymous with “determining” the presence of an agent.

Development of a disease: Pathogenesis of a disease. Progression of a disease from one or more initiating events to expression of disease symptoms. In a particular example, the development of type 1 diabetes can involve an immune-system triggering event that results in production of auto-antibodies to islet cells of the pancreas, and which concludes with glucose metabolic dysfunction.

Immune response: A response of a cell of the immune system, such as a B cell, T cell, or monocyte, to a stimulus. In one embodiment, the response is specific for a particular antigen (an “antigen-specific response”), such as an antigen from a particular rotavirus species or serotype. In one embodiment, an immune response is a T cell response, such as a CD4+ response or a CD8+ response. In another embodiment, the response is a B cell response, and results in the production of specific antibodies, for example antibodies specific for the antigen, such as a rotavirus protein or glycoprotein antigen.

Islets of Langerhans are small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or ascinar) tissue. In vivo, the islets of Langerhans consist of the α cells, β cells, δ cells, and PP cells. Histologically, the islets of Langerhans consist of a central core of β cells surrounded by an outer layer of α cells, δ cells, and PP cells. The islets of Langerhans are sometimes referred to herein as “islets.” In particular examples, type 1 diabetes is associated with the presence of auto-antibodies specific to islet cells.

Preventing or treating a disease: Preventing a disease refers to inhibiting the full development of a disease, for example inhibiting the development of type 1 diabetes in a subject who has been exposed to a potential autoimmunity stimulating condition. Treatment refers to a therapeutic intervention that ameliorates a sign or symptom of a disease or pathological condition after it has begun to develop.

Rotavirus: A common double stranded segmented RNA virus, causal to severe diarrhea in infected subjects. Five species of rotavirus have been identified (species A-E), with species A most commonly infecting humans. Rotavirus strains or serotypes are defined by variations in viral protein 7 (VP7) and viral protein 4 (VP4). VP7 is designated “G”, while VP4 is designated “P”. A typical rotavirus serotype might thus be G1P[8].

Subject: Living vertebrate organisms, a category that includes both human and non-human mammals.

Trimester: The term of a human pregnancy is typically nine months, and which can be divided into thirds or trimesters.

Type 1 diabetes (T1D): An autoimmune disease resultant from self-destruction of insulin-producing beta cells of the pancreas Islets of Langerhans. The associated decrease in insulin production results in dysfunction in glucose metabolism and organ damage. In particular examples, the development of T1D is predicted by the presence of antibodies such as the Glutamic acid decarboxylase (GAD)65, islet antigen 2 (IA2), and zinc transporter 8 (ZnT8) autoantibodies.

Vaccinating: The process or program by which a subject, such as a woman prior to conceiving an embryo, is vaccinated against one or more microbes. Vaccines present microbial antigens to a subject in order to develop immunity against the specific microbe. In particular examples, the process of vaccinating a subject against a microbe, such as a virus, involves a single administration of the viral antigen to the subject. In other examples, multiple (two, three, or more) administrations of the antigen are administered over a specified time period, in order to develop the immunity. Vaccinating a subject can be carried out by any means appropriate to the vaccine formulation. In one non-limiting example, a vaccine to provide immunity against a virus can be formulated for inhalative administration, oral administration, or for intramuscular injection.

### III. Overview of Several Embodiments

Described herein are methods for inhibiting the development of type 1 diabetes mellitus in a subject which include vaccinating the future mother of the subject against at least one rotavirus prior to conception of the subject. In particular embodiments, the vaccination against at least one rotavirus is completed at time prior to pregnancy, such as at least two months prior to the pregnancy.

In particular embodiments, vaccination against least one rotavirus occurs by administering to the future mother a vaccine specific to (e.g. only induces immunity against) at least one rotavirus. In other embodiments, against least one rotavirus occurs by administering to the future mother a vaccine comprising a mixture of a rotavirus vaccine and a vaccine against at least one other microbe.

In particular embodiments, the rotavirus vaccine provides immunity to at least one rotavirus which is a rotavirus from the group consisting of rotavirus species A, rotavirus species B, rotavirus species C, rotavirus species D, and rotavirus species E.

In other particular embodiments, the rotavirus vaccine provides immunity to at least one rotavirus of species A, wherein each rotavirus indicated by the vaccine which has a G serotype selected from the group consisting of: serotype G1, serotype G2, serotype G3, serotype G4, serotype G5, serotype G6, serotype G8, and serotype G9, serotype G10, and serotype G12; and wherein each rotavirus indicated by the vaccine further comprises a P genotype selected from the group consisting of genotype P1, genotype 2A, genotype 3, genotype P4, genotype 5A, genotype P6, genotype P8, and genotype P11.

In particular embodiments, the rotavirus vaccine provides immunity to species Group A. In some embodiments, immunity is provided by a monovalent vaccine containing a single rotavirus with a single human G (G1) and P [8]) type; In other embodiments the immunity is provided by a pentavalent vaccine containing a mixture of 5 rotaviruses with 4 human G types (G1, G2, G3, and G4), one bovine G type, one bovine P-type and one human P-type (P[8]).

Also described herein are methods for decreasing the prevalence of type 1 diabetes mellitus, by vaccinating all women, prior to conception of a child, against at least one species of rotavirus.

In particular embodiments, vaccination of a woman prior to conception is completed at least two months prior to the pregnancy.

In particular embodiments, the at least one rotavirus is selected from the group consisting of rotavirus species A, rotavirus species B, rotavirus species C, rotavirus species D, and rotavirus species E, but more preferably rotavirus species A.

In other embodiments, each of the at least one rotavirus is a rotavirus of species A that has a G serotype selected from the group consisting of: serotype G1, serotype G2, serotype G3, serotype G4, serotype G5, serotype G6, serotype G8, and serotype G9, serotype G10, and serotype G12; and also has a P genotype selected from the group consisting of genotype P1, genotype 2A, genotype 3, genotype P4, genotype 5A, genotype P6, genotype P8, and genotype P11.

In still further embodiments, vaccination includes administering to the woman a vaccine comprising a mixture of an anti-rotavirus vaccine and a vaccine against at least one other microbe.

### IV. Methods of Inhibiting Type 1 Diabetes by Rotavirus Vaccination in Pre-Partum Women

Described herein is the discovery of a statistically significant correlation between anti-rotavirus antibodies and auto-antibodies to pancreatic beta cells in the cord blood of a neonate subject. This discovery indicates that a rotavirus infection during pregnancy can result in an autoimmune triggering event that can lead to T1D in the subject after birth.

Accordingly, described herein are methods for preventing or inhibiting the development of T1D in a subject by vaccination of the mother of the subject against at least one type of rotavirus, prior to conception of the subject.

Vaccination against a microbe can be a single-step procedure, or a multi-step procedure that is carried out by administering a vaccine over the course of a series of weeks or months, but the full immunity intended from a course of vaccination is only achieved with the completion of the course. This disclosure contemplates that the protective effect of rotavirus vaccination in a mother-to-be is not limited to a particular time period (e.g. a particular time period before or during a pregnancy), so long as the vaccination occurs prior to pregnancy.

In particular embodiments, the mother of the subject is vaccinated against at least one rotavirus prior to conception of the subject and the onset of pregnancy. “Prior to conception,” as understood herein, can be any time prior to conception, without limitation. In particular embodiments, the mother-to-be of the subject is months or even years “prior to conception” and any pregnancy.

In particular embodiments, the mother of the subject is intending to become pregnant, and the vaccination is completed no more than 1 year, no more than 10 months, no more than 8 months, no more than 6 months, no more than 4 months, no more than 2 months or no more than 1 month prior to the pregnancy. In particular embodiments, the mother of the subject was previously vaccinated against rotavirus and is provided a booster dose of rotavirus vaccine no later than two months prior to conception of the subject.

### Rotavirus Vaccines

Any vaccine or vaccine course which provides immunity against at least one type of rotavirus can be used in the methods described herein. Such vaccines include those currently approved for use in children (e.g. the ROTATEQ® rotavirus vaccine (Merck), and the ROTARIX® rotavirus vaccine (GlaxoSmithKline); but the vaccines contemplated for use in the described methods also include those that are still in development or are yet to be developed.

Five species of rotavirus are currently known (Rotavirus A, B, C, D, and E). Rotavirus species A is the most prevalent in worldwide human infections. However, a vaccine for use in the disclosed methods, and provided to the subject's mother prior to pregnancy, can produce immunity to one or more of species A, B, C, D, or E. The currently approved vaccines are directed against Rotavirus species Group A.

In addition to different species, rotavirus is classified into different serotypes or genotype based on variations in viral proteins VP7 (“G”) and VP4 (“P”). A typical rotavirus may thus be of species A, and having a G1P[3] serotype. Vaccines currently approved for use in children and contemplated for use in the described methods vary by the rotavirus serotypes in the vaccine.

A vaccine for use in the methods described herein can provide immunity to at least one rotavirus, such as two, three, four, five, or even more distinct rotaviruses. In particular examples, the vaccine is rotavirus-specific, and only provides antigens to produce immunity to at least one rotavirus, and no other types of microbes. In particular examples, the vaccine is directed to a rotavirus of species A that has a serotype of G1 and P[8] for (Rotarix) and G1, G2, G3, G4, G6 and P[7] and P[8] for (RotaTeq) In other examples, the vaccine for use in the described methods is a mixed vaccine, and produces immunity to at least one rotavirus, in addition to one or more other microbe. Such vaccines are not limited by the additional microbe; as long as intended immunity to either microbe is not compromised by the vaccine mixture.

A rotavirus vaccine for use in the described methods can be prepared by any method known to the art, and can be of any vaccine type. In particular embodiments, the vaccine is a killed virus vaccine. In other embodiments, it is a live attenuated vaccine. In still other embodiments, the immune-provoking antigens are not part of a whole virus, but rather are presented in a synthetic framework. It is additionally appreciated that the viral origin of the rotavirus for use in the current methods can be entirely human in some vaccine embodiments, but can be mixtures or “reassortments” of human and non-human rotavirus (see Bhandari et al., The Lancet, Mar. 12, 2014). Vaccines composed of recombinant hybrids of rotavirus and other types of human and/or non-human viruses are also contemplated.

Vaccines for use in the described methods can be prepared and formulated for delivery by any method known in the art. Rotavirus vaccines are commonly formulated for oral administration; however any mode of administration that is effective for vaccine delivery is contemplated. For example, vaccines exist or can be developed for topical administration, subcutaneous administration, intramuscular administration, or administration by inhalation. Suitable preservatives, excipients, and adjuvants are standard in the art and can be used as needed for rotavirus vaccine preparation, storage, and effective delivery, as necessary.

### V. Methods of Decreasing the Prevalence of Type 1 Diabetes

The global incidence of type 1 diabetes (T1D) is increasing, and is a gradually increasing burden on global public health.

Disclosed herein are methods for decreasing the prevalence of T1D in a population. In some embodiments, the methods involve first determining the rotavirus vaccination status of a pre-partum woman or girl. If the woman or girl is determined to not have rotavirus immunity, or to have immunity to a rotavirus strain not commonly found at her current geographic location, the woman or girl is then vaccinated for rotavirus. As described above, rotavirus vaccination is prior to conception of a child, and thus prevention of rotavirus infection during pregnancy, will prevent or inhibit an autoimmune response that can lead to T1D.

Determination of rotavirus vaccination status can be performed by any means known in the art. In particular embodiments, vaccination status is determined by review of vaccination history and/or medical records of the woman or girl in question. In other embodiments, a blood test is performed to detect the presence of rotavirus immunoglobulins in the woman or girl. Particular examples of standard tests to detect specific immunoglobulins include standard ELISA and radioligand binding assays, as described herein.

The determination of vaccination status will inform whether additional steps are needed. If it is determined that the woman or girl is not vaccinated (“non-vaccinated”), then the rotavirus vaccine is administered to the woman or girl. Vaccination is carried out within the time frame and with a vaccine as described above.

In other embodiments, the vaccination status of the “pre-conception” woman or girl only dictates whether the vaccine given is an initial vaccine dose or a booster dose (in the case of a woman or girl who was previously vaccinated). Such booster doses are analogous to a booster dose of other vaccines known to the art that are provided to adults to strengthen immunity previously provided by a childhood immunization.

The following examples are provided to illustrate certain particular features and/or embodiments. These examples should not be construed to limit the disclosure to the particular features or embodiments described.

### EXAMPLES

### Example 1

### Islet Cell Autoantibodies Correlate with Rotavirus Antibodies in Maternal and Neonate Subjects

This example shows the discovery of a positive correlation between rotavirus infection in a pregnant woman and the presence of islet cell autoantibodies in the maternal and neonate cord blood. The results described herein strongly suggest that rotavirus infection during pregnancy provides an initiating event that can lead to development of Type 1 Diabetes (T1D) in a subject.

### Methods

Subjects: Healthy, pregnant women [n=107, mean (range) age 30.7 (19-46) years] from central Israel were enrolled at the time of delivery (January to April 2010, n=76, and January to March 2011, n=43) to coincide with rotavirus seasons] at the Helen Schneider Women's Medical Center. None had a history of Type 1 diabetes in their families. Matched maternal and cord blood samples were collected for 107 of these women at the time of delivery, with first trimester blood available from 20 of the 107 mothers. The Ethical Review Board of the Belinson Medical Center approved the study and all of the women signed an informed consent form.

Antiviral enzyme-linked immunosorbent assays (ELISA): Simian rotavirus strain SA11 (ATCC VR-1565; American Type Culture Collection, Manassas, Va., USA) and CoxB3, isolated from a 2009 clinical sample, were used as antigens to measure antiviral immunoglobulin G (IgG) antibody titres using ELISA. The rotavirus antigen was presented after affinity capture using mouse anti-rotavirus IgG (Enco, Cat. No. 0531, Israel) bound to the ELISA plates, while CoxB3 was bound directly to the plates. Peroxidase-conjugated goat anti-human IgG (cat 109-035-003; Jackson Immuno Research, West Grove, Pa., USA) was used to determine the amount of human IgG bound to the antigens. Supernatant from mock infected MA-104 or human kidney cell cultures, respectively, served as negative controls. The mean OD of negative controls from all assays was determined and clinical samples with OD values two standard deviations above this were considered positive. High, middle and low concentrations of an anti-rotavirus IgG-positive or CoxB3 reference sample were included in all test runs. The relative units (RU) of IgG in all other clinical samples were determined from the regression curve from the mean ODs of the reference from all test runs for each concentration. Acceptance of analytical data from each run was according to Westgard rules. The inter-test CV for the anti-rotavirus IgG standards was 25.06%, while the intra-test CV for 17 replicates of the same sample was 11%. The inter-test CV for the anti-CoxB3 standards was 14. Very recent infections were distinguished from infections that occurred in the past by a seroconversion defined by a 4-fold increase in antibody titre between two successive samples, or acquisition of detectable antibody titres in the follow-up sample. (CDC-recommended guidelines for sero-conversion).

Radioligand binding assay: Glutamic acid decarboxylase (GAD)65, islet antigen 2 (IA2), and zinc transporter 8 (ZnT8) autoantibodies were determined by radioligand binding assay as previously described (Vaziri-Sani et al., Autoimmunity; 43:598-606, 2010). All samples were analysed in triplicate and the mean (range) intra-assay coefficient of variation was 4.5 (0.016-15)%. Our laboratory participated in the Diabetes

Autoantibody Standardization Program workshop, where our GAD65 autoantibody assay showed a sensitivity of 86% and specificity of 93%, and the IA2 autoantibody assay showed a sensitivity of 66% and a specificity of 98%. ZnT8 autoantibodies were not included in the workshop.

Thresholds for Islet Cell Antibody Positivity GAD65 Antibodies:

GAD65 antibodies: The positivity of samples was verified in competition assays using recombinant human GAD65 (200 ng/ml; Diamyd Medical, Stockholm, Sweden) as previously described (Rolandsson et al. Diabetologia, 42: 555-559, 1999). Samples whose binding to radiolabelled GAD65 was reduced by 50% (index ≧0.5) were considered GAD65 antibody-positive. This threshold is equivalent to 40 U/ml.

IA2 autoantibodies: The samples were considered IA2 autoantibody-positive if binding exceeded that of the 98th percentile for healthy controls (30 U/ml).

ZnT8 autoantibodies: The threshold was set at 15 RU/ml for autoantibodies to ZnT8R and 26 RU/ml for ZnT8W based on the 98^{th} percentile observed in 50 healthy human control sera.

Odds ratios: Odds ratios with 95% CIs were calculated according to Bland and Altman (Bland et al., BMJ, 1468:320, 2000) using an online calculator (available at hutchon.net/ConfidORselect.htm).

### Results

The prevalence of GAD65, ZnT8 and IA2 autoantibodies in maternal and cord blood sera from the 107 pregnancies at birth is summarized in Table 1.

TABLE 1

| | | | |
|---|---|---|---|
| GAD65 | ZnT8R | Rotavirus | CoxB3 |

A. Antibody Positive^{a} Cord Bloods

| | | | | | | | | |
|---|---|---|---|---|---|---|---|---|
| GAD65 | 10 | (9.3%) | 0/10 | (0.0%) | 3/10 | (30%) | 4/10 | (40%) |
| ZnT8R | 0/2 | (0.0%) | 2 | (1.9%) | 0/2 | (0.0%) | 0/2 | (0.0%) |
| Rotavirus | 3/26 | (11.5%) | 0/26 | (0.0%) | 26 | (24.3%) | 14/26 | (53.8%) |
| CoxB3 | 5/48 | (10.4%) | 0/48 | (0.0%) | 15/48 | (31.3%) | 48 | (44.9%) |

B. Antibody Positive^{a} Maternal Serum at Delivery

| | | | | | | | | |
|---|---|---|---|---|---|---|---|---|
| GAD65 | 8 | (7.5%) | 0/8 | (0.0%) | 2/8 | (25%) | 4/8 | (50%) |
| ZnT8R | 0/2 | (0.0%) | 2 | (1.9%) | 0/2 | (0.0%) | 0/2 | (0.0%) |
| Rotavirus | 2/11 | (18.2%) | 0/11 | (0.0%) | 11 | (10.3%) | 4/11 | (36.4%) |
| CoxB3 | 4/36 | (11.1%) | 0/36 | (0.0%) | 4/36 | (11.1%) | 36 | (33.6%) |

C. Both Maternal Serum and Cord Blood Antibody Positive^{a}

| | | | | | | | | |
|---|---|---|---|---|---|---|---|---|
| GAD65 | 5 | (4.7%) | 0/5 | (0.0%) | 1/5 | (20%) | 1/5 | (20%) |
| ZnT8R | 0/2 | (0.0%) | 2 | (1.9%) | 0/2 | (0.0%) | 0/2 | (0.0%) |
| Rotavirus | 2/11 | (18.2%) | 0/11 | (0.0%) | 11 | (10.3%) | 4/11 | (37.3%) |
| CoxB3 | 1/32 | (3.1%) | 0/32 | (0.0%) | 4/32 | (13.4%) | 32 | (29.9%) |

^{a}GAD65 Index≧0.5; ZnT8R ≧15 RU; Rotavirus ≧6 RU; and CoxB3 ≧10R

GAD65 antibodies were present in sera from matched cord blood and maternal samples in 5/107 (4.7%) of the pregnancies. In five pregnancies (4.7%) cord blood samples were positive for GAD65 antibodies, while their respective maternal sera tested antibody-negative. GAD65 antibodies were already elevated in the first trimester for two of the 20 pregnancies, where a sample was collected during the first trimester. Cord blood antibody titres for rotavirus and CoxB3 ranged between 3.1 and 96.3 RU, respectively, while maternal antibody titres ranged between 3.0 and 49.6 RU. Altogether 37/107 (35.7%) of pregnancies had ≧6 RU of rotavirus antibodies in their maternal and/or cord blood samples; 24% (26/107) in cord blood samples alone; 10% (11/107) in both maternal and cord blood samples; and none in the maternal sera alone. Moreover, 10/11 pregnancies with detectable rotavirus IgG in maternal samples had rotavirus antibody titres in the cord blood, exceeding the maternal antibody titre by 2.5-5-fold. Antibody titres to CoxB3 enterovirus >10 RU were found in 48% (50/105) of pregnancies in maternal and/or cord blood samples, while 33% (36/105) of maternal serum samples and 46% (48/105) of cord blood samples contained antibody titres to CoxB3 enterovirus >10 RU, and 30% (32/105) of pregnancies were found to have antibodies >10 RU in both samples. Cord blood antibody titres ranged between 3.0 and 21.7 RU, while maternal antibody titres ranged between 3.4 and 16.4 RU.

For the 20 pregnancies where a first trimester maternal serum sample was available, two showed seroconversion indicative of a very recent infection. The first woman acquired a rotavirus antibody titre of 4.3 RU, while the first trimester sample showed no detectable virus antibody titre. No islet cell autoantibodies were detected in her sera, or in the cord blood, while all three samples had CoxB3 IgG titres ranging between 9.9 to 13.1 RU. The second woman acquired a CoxB3 antibody titre of 9.6 RU, while the first trimester sample showed no detectable virus antibody titre. None of the maternal or cord blood samples from this pregnancy contained detectable antibodies against islet cell antigens or rotavirus. The ratio of antibody titres in cord blood compared with corresponding maternal blood at delivery is shown in Table 2.

TABLE 2
Ratio of antibodies in Cord Blood to Maternal serum at delivery.

| Ratio | | GAD65 | Rotavirus | CoxB3 |
|---|---|---|---|---|
| ≧5 | | 0 | 1 | 0 |
| 2.5-4.9 | | 0 | 9 | 0 |
| 0.6-2.4 | | 4 | 22 | 32 |
| <0.6 | | 0 | 0 | 0 |
| Cord | Maternal | | | |
| Pos. | NS. | 5 | 22 | 17 |
| NS. | Pos. | 3 | 0 | 2 |
| NS. | NS. | 95 | 56 | 54 |

| | | | |
|---|---|---|---|
| Total number | 107 | 107 | 105 |

NS. = Not significant (GAD65) or below detection (Rotavirus, CoxB3).

When comparing serum samples positive for GAD65 antibodies with sera containing ≧6 RU for rotavirus IgG or >10 RU of CoxB3 IgG, we observed a significant odds ratio of 6.89 (95% CI 1.01-46.78) for pregnancies where both cord blood and maternal serum at birth were positive for GAD65 antibodies and rotavirus IgG.

### Discussion

The present study showed that islet cell autoantibodies can be detected at delivery in healthy mothers and/or their offspring. None of the mothers had a family history of diabetes. Therefore, the findings should be related to an environmental event occurring during pregnancy, such as a viral infection. Development of islet cell autoimmunity, with the characteristic autoantibodies subsequent to such a viral infection, is likely to occur weeks or months after the virus has cleared the system and virus concentrations drop below the levels of detection by molecular assays. Serological assays for the presence of antibodies to rotavirus and enterovirus are one way to circumvent this difficulty.

We found a significant correlation (odds ratio 6.89; 95%, CI 1.01-46.78) between GAD65 antibodies and rotavirus antibodies in pregnancies where both maternal and cord blood samples were positive for GAD65 antibodies and both had ≧6 RU of rotavirus IgG. This odds ratio dropped to 4.85, but was no longer significant (95% CI 0.54-51.81) when the threshold for rotavirus IgG titres was set to ≧10 RU. Notably, four of the GAD65 antibody-positive cord blood samples with GAD65 antibody-negative mothers had >10 RU of CoxB3 antibody and the fifth had 9.4 RU.

The finding of GAD65 antibodies in the cord blood of five neonates without the presence of these antibodies in the corresponding mother, and antibody titres to rotavirus and/or enterovirus 2.5-5-fold higher than in the corresponding maternal samples, may be indicative of an independent humoral response (Kapur et al., Fanaroff and Maritin's Neonatal-Perinatal Medicine Diseases of the Fetus and Infant, 9th edn. St. Louis: Elsevier, 2011), to an insult of the fetal pancreas. Similar findings have been reported for GAD65 antibodies (Elfving et al. Horm Metab Res; 39: 790-796, 2007; Merbl et al., J Clin Invest; 117: 712-718, 2007) and independent cellular fetal immune responses to viral infections have been reported (Elbou Ould et al., Pediatr Res; 55: 280-286, 2004; Hermann et al., Blood; 100: 2153-2158, 2002). A higher frequency of GAD65 antibody-positive cord blood samples compared with those found in a study in Finland (Hamalainen et al., Diabetes Metab Res Rev; 18:57-63, 2002) and the Diabetes Autoimmunity Study in the Young cohort study in the USA (Stanley et al., Diabetes Care; 27: 497-502, 2004) may be attributable to the fact that the samples in the present study were collected specifically from children who were born during peak viral seasons. Another explanation could be related to different thresholds for GAD65 antibody positivity. GAD65 antibody positivity in the present study was confirmed in a specific competition assay (Rolandsson et al., Diabetologia; 42: 555-559, 1999).

Paired samples with antibodies in both cord blood/maternal samples suggest transplacental transfer of antibodies. Cord blood antibody levels tend to be higher (˜60%) than those of the mother at the time of delivery, possibly as a result of active transport of IgG across the placenta (Palfi et al., Am J Reprod Immunol; 39: 24-26, 1998; Simister et al., Vaccine; 21: 3365-3369, 2003; Lee et al., Monogr Allergy; 19: 108-121, 1986) and/or haemodilution in the mother. It was evaluated whether cord blood samples with 2.5-5-fold higher rotavirus and/or GAD65 antibody levels as compared with the respective maternal sample showed corresponding differences in tetanus toxoid antibody levels and total IgG levels in cord blood and mothers. Although tetanus toxoid antibody levels in cord blood were on a mean (range) of 1.3 (0.3-4.1)-fold higher than the maternal antibody levels, no correlation was found between the tetanus toxoid antibody ratios and the aforementioned ratios between cord blood and maternal antibodies to rotavirus and GAD65 antibody. Similar results were observed for total IgG. It is therefore unlikely that the observed higher rotavirus and GAD65 antibody levels in cord blood were caused by active transplacental transmission or haemodilution in the mother, but are more likely to have resulted from an independent fetal immune response to viral infection and islet cell autoantigens.

The present study differs from the TEDDY study (Lee et al., Diabetologia; 56: 1705-1711, 2013) in that the present study investigated pre and neonatal conditions, while the TEDDY study investigated post-natal conditions. Another difference is our concept of viral infection as an initiating trigger of the autoimmune process leading to Type 1 diabetes, whereas the TEDDY study investigated whether viral infections immediately preceded the conversion of preclinical to clinical childhood Type 1 diabetes. The observed lack of correlation of enteroviral antibodies and islet cell autoantibodies is consistent with other studies (Fuchtenbusch et al., J Autoimmun; 17: 333-340, 2001; Viskari et al., Diabetes; 51: 2568-2571,2002). The significance of the findings of the present pilot study needs to be confirmed.

In conclusion, the present findings may support the hypothesis that maternal rotavirus infections during pregnancy may damage the fetal islet cells and trigger the cascade of events leading to Type 1 diabetes.

### Example 2

### Vaccination Against Rotavirus to Prevent Type 1 Diabetes

This example illustrates that vaccination of women prior to conception of a child has a protective effect against T1D development in the child.

Methods are as described in Example 1 where applicable, except as indicated herein. Subjects are selected from a similar demographic group as in Example 1, with no family history of T1D, and no record of rotavirus vaccination. Further, subjects are not pregnant, but intend to become so.

Once chosen, subjects are vaccinated against rotavirus with any publicly available licensed rotavirus vaccine (e.g. the ROTATEQ® rotavirus vaccine (Merck), the ROTARIX® rotavirus vaccine (GlaxoSmithKline), and the rotavac vaccine). Vaccination courses are completed for each subject prior to pregnancy.

Vaccinated women are tracked for the presence of rotavirus and islet autoimmune response as described above. Serum samples are taken from women following vaccination, during pregnancy, and post-partum. Cord serum is also sampled for detection of rotavirus and GAD65 (islet autoimmune) antibodies. Antibody detection is as described above. The presence of rotavirus and GAD65 antibodies in the vaccinated cohort is compared with historical data (for example, that presented in Example 1). In comparison to cord serum sampled from neonates of non-vaccinated mothers, the cord serum of the vaccinated cohort displays a statistically significant decrease in the number of subjects exhibiting the presence of GAD65 antibodies.

### Example 3

### Vaccination Against Rotavirus to Reduce the Prevalence of Type 1 Diabetes (T1D)

This example demonstrates that determination of the rotavirus vaccination status of a pre-partum woman can reduce the prevalence of T1D.

Subjects are similar to those described above, with the condition that none are currently pregnant. Each subject is interviewed, and medical records reviewed, to determine vaccination history. Those women who have previously completed a rotavirus vaccination course are determined by medical records. If no records exist, vaccination status is determined by detection of anti-rotavirus antibodies by ELISA, as described above.

Those women who test negative for rotavirus immunity (and who are considered “non-vaccinated”) are administered a course of the ROTATEQ® pentavalent rotavirus vaccine (Merck) or the monovalent ROTARIX® rotavirus vaccine (GlaxoSmithKline), according to manufacturer's instructions.

Women enrolled in the study are monitored for any pregnancies. During pregnancy, the presence of rotavirus infection and anti-pancreatic beta cell antibodies are monitored in the pregnant woman and the cord blood of the newborn baby. In comparison to historical controls (for example, that presented in Example 1), the rotavirus-vaccinated women in the present study have a lower incidence of rotavirus infection and a lower incidence of anti-beta cell antibodies than in the historical controls. This decrease indicates that rotavirus vaccination inhibits or prevents rotavirus infection during pregnancy, and an associated auto-immune triggering event.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for inhibiting or preventing the development of type 1 diabetes mellitus in a subject comprising:
vaccinating a future mother of the subject against at least one rotavirus prior to conception of the subject;
thereby inhibiting the development of the type 1 diabetes.

2. The method of claim 1, wherein, the vaccinating is completed prior to conception.

3. The method of claim 2, wherein the vaccinating is completed at least two months prior to conception.

4. The method of claim 1, wherein the vaccinating comprises administering to the future mother a vaccine specific to at least one rotavirus.

5. The method of claim 1, wherein the at least one rotavirus is selected from the group consisting of rotavirus species A, rotavirus species B, rotavirus species C, rotavirus species D, and rotavirus species E.

6. The method of claim 1, wherein each of the at least one rotaviruses comprises a species Group A rotavirus comprising a G serotype selected from the group consisting of: serotype G1, serotype G2, serotype G3, serotype G4, serotype G5, serotype G6, serotype G8, and serotype G9, serotype G10, and serotype G12; and further comprises a P genotype selected from the group consisting of genotype P1, genotype 2A, genotype P3, genotype P4, genotype 5A, genotype P6, genotype P8, and genotype P11

7. The method of claim 1, wherein the vaccinating comprises administering to the future mother a vaccine comprising a mixture of an anti-rotavirus vaccine and a vaccine against at least one other microbe.

8. The method of claim 1, wherein the vaccinating comprises administering to the future mother a rotavirus vaccine selected from the group consisting of a monovalent vaccine comprising a single rotavirus with a G1 serotype and P[8] genotype, a pentavalent vaccine comprising a mixture of 5 rotaviruses with 4 human G serotypes of G1, G2, G3, and G4, and one bovine G type, one bovine P-type, and one human P-type (P[8]), and any formulation of rotavirus vaccine licensed for use in children.

9. A method for decreasing the prevalence of type 1 diabetes mellitus comprising:
determining if a woman of child-bearing age has been vaccinated against rotavirus; and
prior to conception, vaccinating non-vaccinated women against at least one rotavirus;
thereby decreasing the prevalence of type 1 diabetes mellitus.

10. The method of claim 9, wherein determining if a woman of child-bearing age has been vaccinated against rotavirus comprises determining the presence anti-rotavirus antibodies in the blood of the woman.

11. The method of claim 9, wherein the vaccinating is completed prior to conception.

12. The method of claim 9, wherein the vaccinating is completed at least two months prior to the conception.

13. The method of claim 9, wherein the at least one rotavirus is selected from the group consisting of rotavirus species A, rotavirus species B, rotavirus species C, rotavirus species D, and rotavirus species E.

14. The method of claim 9, wherein each of the at least one rotaviruses comprises a rotavirus of species A comprising a G serotype selected from the group consisting of: serotype G1, serotype G2, serotype G3, serotype G4, serotype G5, serotype G6, serotype G8, and serotype G9, serotype G10, and serotype G12; and further comprises a P genotype selected from the group consisting of genotype P1, genotype 2A, genotype 3, genotype P4, genotype 5A, genotype P6, genotype P8, and genotype P11.

15. The method of claim 9, wherein the vaccinating comprises administering to the woman of child bearing age a rotavirus vaccine selected from the group consisting of a monovalent vaccine comprising a single rotavirus with a G1 serotype and P[8] genotype, a pentavalent vaccine comprising a mixture of 5 rotaviruses with 4 human G serotypes of G1, G2, G3, and G4, and one bovine G type, one bovine P-type, and one human P-type (P[8]), and any formulation of rotavirus vaccine licensed for use in children.

16. The method of claim 9, wherein the vaccinating comprises administering to the woman a vaccine comprising a mixture of an anti-rotavirus vaccine and a vaccine against at least one other microbe.

17. The method of claim 9, wherein if the woman of child bearing age has been previously vaccinated, administering an adult-dose booster rotavirus vaccine.

18. A method for decreasing the prevalence of type 1 diabetes mellitus comprising:
prior to conception of a baby, vaccinating women of child bearing age against at least one rotavirus;
thereby decreasing the prevalence of type 1 diabetes mellitus.

19. The method of claim 18, wherein the vaccinating comprises administering to the woman of child bearing age a rotavirus vaccine selected from the group consisting of a monovalent vaccine comprising a single rotavirus with a G1 serotype and P[8] genotype, a pentavalent vaccine comprising a mixture of 5 rotaviruses with 4 human G serotypes of G1, G2, G3, and G4, and one bovine G type, one bovine P-type, and one human P-type (P[8]), and any formulation of rotavirus vaccine licensed for use in children.
